(19) United States
(12) Patent Application Publication
(10) Pub. No.: US 2015/0328155 A1
(43) Pub. Date: Nov. 19, 2015

(54) GEL COMPOSITION CAPABLE OF USE AS A DRUG DELIVERY DEVICE AND METHOD OF USE THEREOF

(71) Applicant: CMAB HOLDINGS, LLC, Fairhope, AL (US)

(72) Inventors: Christopher A. Burgess, Fairhope, AL (US); Josh Smith, Mobile, AL (US); Jared Taylor, Mobile, AL (US)

(73) Assignee: Heritage Compounding Pharmacy

(21) Appl. No.: 14/715,980

(22) Filed: May 19, 2015

Related U.S. Application Data
(60) Provisional application No. 62/000,277, filed on May 19, 2014.

(51) Int. Cl. A61K 9/12; A61K 47/36; A61K 47/38; A61K 9/00

(52) U.S. Cl. CPC A61K 9/124; A61K 9/0043; A61K 47/36; A61K 47/38

(57) ABSTRACT

The present invention provides a gel composition intended for use as a drug delivery device, particularly for use in the sinus cavity. The gel composition can include a gelling agent that includes at least one polysaccharide, a propellant in the form of a sterile gas, and optionally, at least one active ingredient. The polysaccharide can be selected from the group consisting of hydroxyethyl cellulose, hydroxypropyl cellulose, carboxymethyl cellulose, carrageenans, and combinations thereof.

### CROSS-REFERENCE TO RELATED APPLICATIONS

The present application claims priority to U.S. Provisional Patent Application No. 62/000,277, filed May 19, 2014, which is incorporated herein by reference in its entirety.

### FIELD OF THE INVENTION

The present invention relates to devices suitable for packing and/or drug delivery. In particular, the present invention relates to devices suitable for delivering drugs, for example, to the sinus cavity.

### BACKGROUND OF THE INVENTION

Nasal packing is the application of packs to the nasal cavities. A common purpose of nasal packing is to control bleeding following surgery to the septum or nasal reconstruction, to prevent synechiae (adhesion) or restenosis, and to treat epistaxis (nose bleeding). Packing can also provide support to the septum after surgery. Additionally, packing can provide delivery of drugs to the patient, which can thereby provide patient comfort and/or antibiotics to an intended location, and can sometimes alleviate the need for an invasive surgery. See, e.g., Verim et al., Role of Nasal Packing in Surgical Outcome for Chronic Rhinosinusitis With Polyposis, The Laryngoscope 00: Month, pp. 1-7, 2013, herein incorporated by reference.

Numerous materials have been proposed in the prior art for use as dental and biomedical forms for adsorbing or removing body fluids. Conventional non-biodegradable packings (e.g., packings consisting of gauze and cotton) have several disadvantages: the fluid absorption capacity of the material is relatively low, the structure is relatively fragile and individual threads or fibers can break off, and failure to remove the material from the body after surgery can lead to serious complications. Removing the pack when the wound is still fresh can damage the nasal cavity again and cause the patient discomfort. Certain hydrophilic synthetic materials intended for biomedical applications have improved properties when compared to conventional properties; however, these materials are still not biodegradable. For example, see the cross-linked polyurethane-based hydrogels disclosed in U.S. Pat. No. 3,903,232; U.S. Pat. No. 3,961,629; and U.S. Pat. No. 4,550,126.

In order to prevent the undesired effects of non-biodegradable materials, biodegradable sponges or absorbing foams comprising materials of a natural source (e.g., gelatin, proteins, chitin, cellulose, and polysaccharides) have been suggested. See, e.g., the discussion of biodegradable nasal packings in Yan M, Zheng D, Li Y, Zheng Q, Chen J, et al. (2014) Biodegradable Nasal Packings for Endoscopic Sinonasal Surgery: A Systematic Review and Meta-Analysis. PLoS ONE 9(12): e115458. doi:10.1371/journal.pone.0115458; and in GMS Current Topics in Otorhinolaryngology—Head and Neck Surgery available at http://www.egms.de/static/en/journals/cto/2011-8/cto000054.shtml; each of which is herein incorporated by reference. However, many of these materials as previously used lacked the required mechanical strength to function as desired. In addition, properties of natural polymers can be difficult to control and may have batch variations, they can be more expensive than synthetic materials, and biological hazards can be associated with the use of biodegradable materials of natural sources.

Synthetic materials that are capable of being absorbed by the body have been disclosed in several patent applications. For example, U.S. Pat. No. 3,902,497 and U.S. Pat. No. 3,875,937 disclose surgical dressings of bio-absorbable polymers of polyglycolic acid (PGA). However, such materials are relatively hard, brittle and not resilient, and, therefore, not suited for application in many medical situations.

U.S. Appl. Publ. No. 2006/0008419, for example, discloses an absorbent foam comprising a biodegradable synthetic polymer, which polymer comprises —C(O)—O— groups in the backbone of the polymer (e.g., polyurethane and/or polyester units combined with polyethers). However, polyesters may be irritating agents in a mucous membrane or cavity. The invention described therein also discloses the use of vinyl alcohol, which may also be irritating. In addition, the absorbent foam discussed in 2006/0008419 does not possess a thixotropic effect. A thixotropic effect enables a gel packing product to reach a rigidity desired for packing in the sinus cavity while still maintaining a malleability that allows the gel to be applied in a proper manner. Furthermore, 2006/0008419 discloses a process that takes place in the sinus cavity, which can make it difficult to determine the final viscosity of the product before administration.

U.S. Appl. Publ. No. 2003/0187381, for example, provides a bioabsorbable foam sodium hyaluronate and carboxymethylcellulose. However, the carboxymethylcellulose-based gel produces a relatively thin gel that does not provide optimal application as it tends to break down faster than desired.

U.S. Appl. Publ. No. 2010/0016267, for example, discloses pharmaceutical compositions for delivering a drug to a sinus comprising a drug combined with a high viscosity liquid carrier material (HVLCM) such as sucrose acetate isobutyrate (SAIB), together with any other suitable ingredients such as polymers, excipients, solvents, stabilizers, etc. However, the product described therein involves the use of a poloxamer gel. Poloxamer gels require storage at a low temperature in order to maintain a viscosity useful in application to the sinus cavity. The gel must then be warmed to attain a gel-like state in which the gel can adhere to the sinus cavity. This can provide only a narrow window of opportunity for a physician to apply the product. It also can be difficult to add enough thickening powder to a product that includes a poloxamer gel, thereby limiting the time that these products remain in the sinus cavity. Furthermore, 2010/0016267 discloses a process that takes place in the sinus cavity, which can make it difficult to determine the final viscosity of the product before administration.

There is a continuing need for new packing and/or drug delivery devices that exhibit advantageous properties without producing undesirable side effects. In particular, biodegradable packing and/or drug delivery devices that are capable of forming a thick gel with high absorption capacities and physical properties beneficial for use in various medical applications are desired.

### SUMMARY OF THE INVENTION

The present invention provides compositions useful as a drug delivery device, particularly for use in the sinus cavity. The compositions can be in a gel form and can include a gelling agent comprising at least one polysaccharide, a propellant comprising a sterile gas, and optionally, at least one active ingredient.

In various embodiments of the present invention, a gel composition for use in the sinus cavity can be provided comprising a gelling agent comprising a first polysaccharide, a second polysaccharide, wherein the second polysaccharide is Kappa-carrageenan, optionally a propellant in the form of a sterile gas, and optionally at least one active ingredient. The first polysaccharide can be selected from the group consisting of hydroxyethyl cellulose, hydroxypropyl cellulose, carboxymethyl cellulose, and combinations thereof. In certain embodiments, the first polysaccharide is hydroxyethyl cellulose. The first polysaccharide can be present in an amount of at least about 5 dry weight percent, at least about 10 dry weight percent, at least about 20 dry weight percent, at least about 40 dry weight percent, or at least about 60 dry weight percent. In various embodiments, the Kappa-carrageenan can be present in an amount of at least about 5 dry weight percent, at least about 10 dry weight percent, at least about 20 dry weight percent, at least about 40 dry weight percent, or at least about 60 dry weight percent. In some embodiments, the first polysaccharide and the Kappa-carrageenan can be present in about a 1:1 ratio by weight.

In some embodiments of the present invention, the propellant can comprise carbon dioxide gas. The carbon dioxide gas can be an in situ reaction product of an acid component and a base component. In certain embodiments, the base component can comprise sodium bicarbonate (NaHCO₃). In various embodiments, the acid component can comprise at least one of ascorbic acid, citric acid, and sorbic acid. In a preferred embodiment, the acid component can be ascorbic acid. The base component and the acid component can be initially present in the gel composition in stoichiometric amounts such that the amount of base component is sufficient to react with substantially all of the acid component. In certain embodiments, the base component and the acid component can be initially present in the gel composition in stoichiometric amounts such that the amount of base component is sufficient to react with only a desired portion of the acid component.

In various embodiments, the gel composition can further comprise at least one surfactant. The surfactant can be present in an amount of about 0.0001 to about 5.0 dry weight percent.

In various embodiments of the present invention, a composition for use in a sinus cavity is provided, comprising a first polysaccharide in solid form, a second polysaccharide in solid form, wherein the second polysaccharide is Kappa-carrageenan, and optionally one or both of an acid in solid form and a base in solid form. In some embodiments, all of the first polysaccharide, the second polysaccharide, and the acid and/or base can be in powdered form.

A method of providing a sterilized gel composition is also provided herein. The method can comprise providing a gelling agent mixture comprising at least a first solid polysaccharide and second solid polysaccharide, wherein the second solid polysaccharide is Kappa-carrageenan, sterilizing the gelling agent, solubilizing the gelling agent with sterile water to form a gelling solution, and adding a propellant to the gelling solution to form a sterilized gel composition. The propellant can comprise carbon dioxide gas, for example. In some embodiments, the carbon dioxide gas is a reaction product of an acid component and a base component, wherein the base component and the acid component are initially present in the gel composition in stoichiometric amounts such that the amount of base component is sufficient to react with the desired amount of acid component.

In some embodiments, the step of adding a propellant to the gelling solution comprises mixing an acid precursor in powder form and a base precursor in powder form with the gelling agent mixture before solubilizing the gelling agent mixture. As such, the sterile water acts to form the gel and promote the reaction between the acid precursor and the base precursor that forms the propellant. In certain embodiments, the step of adding a propellant to the gelling solution comprises mixing an acid precursor in powder form with the gelling agent mixture and mixing a base component in powder form with the sterile water to form a base precursor solution, wherein the base precursor solution is used to solubilize the gelling agent mixture and thereby form the gel and allow for the reaction between the acid precursor and the base precursor that forms the propellant.

A sinus packing method is also provided herein wherein the method can comprise introducing into a sinus cavity a composition comprising a first polysaccharide and optionally a second polysaccharide, wherein if present, the second polysaccharide is Kappa-carrageenan. In various embodiments, the first polysaccharide can be hydroxyethyl cellulose, for example. Such a method can encompass any of the compositions described herein.

A kit for the preparation of a composition for use in a sinus cavity is also provided herein. The kit can comprise one or more containers containing a first polysaccharide in solid form, a second polysaccharide in solid form, wherein the second polysaccharide is Kappa-carrageenan, and one or both of an acid and a base, and instructions for mixing the one or more containers with an aqueous medium to form a gel including a propellant. In various embodiments, the kit includes an acid in solid form and the acid is separate from the first polysaccharide in solid form and the second polysaccharide in solid form before mixing. In some embodiments, the kit includes an acid in solid form and the acid is mixed in a single container with the first polysaccharide in solid form and the second polysaccharide in solid form prior to forming a gel with a propellant.

The kit can comprise a base component and an acid component. In various embodiments, the acid is in solid form and can be separate from the base prior to mixing. In certain embodiments, the base can be in solid form. In some embodiments, the base can be an aqueous solution. In various embodiments, the instructions can include mixing the contents of the one or more containers with an aqueous solution of the base, such that the gel is formed and the acid reacts with the base component to form the propellant in the gel.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water).

In the present invention, gel compositions are provided, wherein the gel can be capable for use as a drug delivery device and/or packing material in an internal body cavity. In particular, the various compositions provided herein can be used in the sinus cavity to prevent, for example, adhesions within the nasal cavities, control minor bleeding, minimize edema, and/or eliminate the need for post-op packing. The gel can be stiff enough to provide a stinting effect. Additionally, since the mixtures can be provided in the form of a gel-based foam, they can conform to the sinus cavity and provide a physical barrier. Furthermore, the compositions can be biodegradable and therefore dissolve over time.

In various embodiments of the present invention, a gel composition can comprise a gelling agent, optionally a propellant, and optionally an active ingredient. The gel composition can further comprise a surfactant to adjust foam stability and thereby tailor the gel based on the desired length of time the gel stays in the nasal cavity.

In various embodiments, a gelling agent of the compositions described herein can be a substance with sufficient surface tension to contain air in pressurized micelles. In some embodiments, the gel composition can comprise a water- or alcohol-based solution of a gelling agent. Various embodiments of the compositions of the present invention comprise a gelling agent comprising at least one polysaccharide. The gelling agent can be present in an amount of at least about 30 dry weight percent, at least about dry 40 percent, at least about 60 dry weight percent, at least about 80 dry weight percent, or at least about 90 percent of the dry weight of the gel composition. In certain embodiments, the gelling agent can comprise about 30 to about 99 percent, or about 50 to about 99 percent of the dry weight of the composition.

In some embodiments, the composition can comprise at least one polysaccharide. More particularly, the composition can comprise at least a first polysaccharide and a second, different polysaccharide. Exemplary polysaccharides that may be used herein are further described; however, such description should not be viewed as limiting since the invention can encompass the use of similar materials as may be evident in light of the totality of the disclosure provided herein.

In various embodiments of the composition, the gelling agent can comprise a polysaccharide selected from the group consisting of hydroxyethyl cellulose, hydroxypropyl cellulose, carboxymethyl cellulose, and combinations thereof. In various embodiments, the gel composition comprises hydroxyethyl cellulose. Hydroxyethyl cellulose is a non-ionic cellulose derived polysaccharide that can be useful as a thickening and gelling agent. It can improve drug absorption via hydrophilization, for example. Hydroxyethy cellulose is also highly soluble and non-toxic. In various embodiments of the gel composition, a first polysaccharide can be present in an amount of at least about 3 dry weight percent; at least about 5 dry weight percent; at least about 10 dry weight percent; at least about 25 dry weight percent; or at least about 50 dry weight percent of the total weight of the composition. In certain embodiments, the first polysaccharide (e.g., hydroxyethyl cellulose) can be present in an amount of about 10 to about 90 dry weight percent, about 20 to about 60 dry weight percent, or about 25 to about 50 dry weight percent.

In various embodiments of the gel composition of the present invention, the gelling agent can comprise a second polysaccharide, for example a carrageenan. Carrageenans are linear sulphated polysaccharides that have several desirable properties including solubility, gel formation, and thixotropicity. Commercial carrageenan is divided into three classifications based on the number and distribution of the sulfate ester substitution on the galactose subunit, with Kappa-carrageenan (κ-carrageenan) being the least substituted and Lambda-carrageenan (λ-carrageenan) being the most substituted. Kappa-carrageenan is soluble in cold water and produces a stiff gel in aqueous solution. In a preferred embodiment, the gel composition comprises Kappa-carrageenan. The gel can be stiff enough to provide a stinting effect that increases with the increased concentration of Kappa-carrageenan. A physician can choose the amount of Kappa-carrageenan necessary for a particular application. For example, in some embodiments, Kappa-carrageenan can be present in an amount of about 1-10 weight percent, or about 1-5 dry weight percent based on the total weight of the composition. As used herein, the total weight of the composition refers to the weight of the composition after, for example, water or a solution of a base component has been added to a dry mixture of the composition described herein. In certain embodiments, the gel composition comprises at least about 3 dry weight percent; at least about 5 dry weight percent; at least about 10 dry weight percent; at least about 25 dry weight percent; or at least about 50 dry weight percent of the second polysaccharide (e.g., Kappa-carrageenan) based on the total dry weight of the composition. In certain embodiments, the second polysaccharide (e.g., Kappa-carrageenan) can be present in the composition in an amount of about 10 to about 90 dry weight percent, about 20 to about 60 dry weight percent, or about 25 to about 50 dry weight percent.

In various embodiments, the gel composition comprises a gelling agent comprising a first polysaccharide (e.g., hydroxyethyl cellulose) combined with Kappa-carrageenan The hydroxyethyl cellulose/Kappa-carrageenan mixture has a thixotropic effect that is unique. Under sheering stress (e.g., when pushing the gel through a syringe as a physician would do in the application of the gel composition), the gels comprising this particular polysaccharide mixture becomes thinner, thereby moving through the syringe and apparatus easily. Upon the cessation of that sheering stress, the gels of the present invention maintain their rigidity. Therefore, the physician can get a stinting effect inside the nasal cavity without increasing the difficulty in the application of the gel through the apparatus.

In various embodiments of the present invention, the gel composition comprises at least about 1 dry weight percent, at least about 10 dry weight percent, at least about 25 dry weight percent or at least about 50 dry weight percent of hydroxyethyl cellulose and at least about 1 dry weight percent, at least about 10 dry weight percent, at least about 25 dry weight percent or at least about 50 dry weight percent of κ-carrageenan. The gel composition can have, for example, about 40 dry weight percent, about 60 dry weight percent, about 80 dry weight percent, or about 99 dry weight percent of a mixture of κ-carrageenan and hydroxyethyl cellulose. The ratio of hydroxyethyl cellulose (or other polysaccharide) to Kappa-carrageenan in the gel composition can range from about 0.15:1 to about 1:0.15, more particularly about 0.5:1 to about 1:0.5, or about 1:1. For example, the gel composition can comprise a gelling agent comprising about 80 to about 85 dry weight percent hydroxyethyl cellulose and about 15 dry weight percent κ-carrageenan, or about 60 to about 65 dry weight percent hydroxyl ethylcellulose and about 35 dry weight percent κ-carrageenan. Although a 1:1 mixture of κ-carrageenan and hydroxyethylcellulose can be preferred, other mixtures can provide certain qualities that can be desirable in different situations. For example, the amount of each polysaccharide present can be directly proportional to the viscosity of the product. This can provide individualized care based on patient needs as determined by the physician at the time of administration.

In various embodiments of the present invention, the gelling agent of the composition can be provided in a solid form. Each polysaccharide in a gelling agent mixture can be characterized as granules, particulates and/or fine powders, for example. The substantially dry mixture of polysaccharides can have a moisture content of less than about 5 weight percent, less than about 1 weight percent, or less than about 0.5 weight percent, for example.

In various embodiments, the gel composition comprises a propellant and/or one or more propellant precursors. Suitable propellant precursors can comprise an acid component and a base component. The base component can comprise at least one of a carbonate material and a bicarbonate material. In some embodiments, the acid can be selected from the group consisting of ascorbic acid (vitamin C), citric acid, sorbic acid, and combinations thereof. Suitable propellants can comprise physiologically compatible gases, such as carbon dioxide (CO₂), which can be derived, for example, from the reaction of ascorbic acid with sodium bicarbonate (NaHCO₃), which is commercially available as a sterile product. This reaction can remove the use of potentially non-sterile ambient air from being employed as the air portion of what is intended to be a sterile foam. In a preferred embodiment, the propellant can comprise citric acid as its pKₐ closely matches the pK_{b} of sodium bicarbonate.

In various embodiments, the dry weight percent of propellant precursor included in the gel can be about 0.1% to about 20%, about 0.1% to about 10%, or about 1% to about 5% based on the total weight of the composition. In some embodiments, the gel can comprise at least about 0.1 dry weight percent, at least about 1 dry weight percent, at least about 5 dry weight percent, at least about 10 dry weight percent, or at least about 15 dry weight percent of a propellant precursor. The gel composition can comprise at least about 0.1 to at least about 15, at least about 0.1 to at least about 5, or at least about 0.1 to at least about 1 dry weight percent of an acid component. The gel composition can comprise at least about 0.1 to at least about 15, at least about 0.5 to about at least about 10, or at least about 1 to at least about 5 dry weight percent of a base component. The propellant precursor can comprise a stoichiometric amount of the base material with respect to the acid component such that the base component is sufficient to react with all of the acid component to form carbon dioxide. In a preferred embodiment, after mixing the composition with a liquid (e.g., water), the gel composition can comprise about 1 to about 10% by total weight of a base component and about 0.5% to about 2.5% by total weight of an acid component. A physician can adjust the amount of base component added to the composition to achieve a desired volume of foam from the reaction of the base component with the acid component. For example, a larger amount of base component will allow for more of the acid component to undergo the reaction that forms CO₂ gas (i.e., more foam will be produced). As the amount of base component used in the composition increases, the amount of water in the hydrated composition can decrease. In various embodiments, the CO₂ generated can account for about 20 to about 60, about 25 to about 50, or about 30 to about 45 percent of the total volume of the hydrated composition. In a preferred embodiment, the CO₂ generated can account for about 40 percent of the total volume of the hydrated composition.

In various embodiments of the present invention, the one or more propellant precursors of the composition can be provided in a substantially dry and solid form. The acid component and/or base component can be characterized as granules, particulates and/or fine powders, for example. The substantially dry acid component and/or base component can have a moisture content of less than about 5 weight percent, less than about 1 weight percent, or less than about 0.5 weight percent, for example.

In some embodiments, both the first propellant precursor (e.g., the acid component) and the second propellant precursor (e.g., the base component) can be in a substantially dry and solid form and each propellant precursor can be stored separately from the gelling agent, which can also be in a substantially dry and solid form. All of the substantially dry ingredients can be mixed together with water before use. In various embodiments, only one propellant precursor (e.g., an acid component) in a substantially dry and solid form can be pre-mixed with the gelling agent. A second propellant precursor (e.g., a base component) can be mixed with the gelling agent and the first propellant precursor separately (e.g., upon use). In some embodiments, an aqueous solution of a second propellant precursor is mixed with a substantially dry mixture comprising a gelling agent and a first propellant precursor. Upon mixing the aqueous solution of the second propellant precursor with the substantially dry mixture, the first propellant precursor reacts with the second propellant precursor to form the propellant which in turn causes the gel composition to foam and be in a state ready for application.

In some embodiments, a surfactant can be added to the gel composition. Without being limited by theory, a positively charged surfactant can increase foam stability and nonionic and negatively charged surfactants can reduce foam stability. In some embodiments, the surfactant can be a water miscible surfactant. In various embodiments, the gel composition can comprise a surfactant selected from the group consisting of an anionic surfactant such as sodium dodecyl sulfate and/or sodium stearate; a nonionic surfactant such as triton x-100, sorbitan monostearate/monooleate, polyethylene glycol, and/or polysorbate; a cationic surfactant such as quartenary ammonium compounds; and combinations thereof. In various embodiments, a substantially dry and solid form of a surfactant can be mixed into the composition comprising the gelling agent. In some embodiments, an aqueous solution comprising at least one surfactant can be mixed with a substantially dry composition comprising a gelling agent in solid form.

In various embodiments, a surfactant can be present in an amount of about 0.0001% to about 5% by dry weight of the composition, or about 0.0001% to about 1% by dry weight of the composition, or about 0.0001% to about 0.5% by dry weight of the composition. The addition of a surfactant can necessitate adjustment of the total dry weight percent of the gelling agent in the composition. For example, in certain embodiments a surfactant can improve drug delivery or adherence of the gel product, but might also lessen the viscosity of the gel composition. As such, the composition can be adjusted to compensate for the change in viscosity.

In certain embodiments, the gel composition can further comprise at least one active ingredient such as steroids, antibiotics, and/or antifungals as desired. In various embodiments, a substantially dry and solid form of an active ingredient can be mixed into a substantially dry gel composition comprising a gelling agent. In some embodiments, an aqueous solution comprising at least one active ingredient can be mixed with a substantially dry composition comprising a gelling agent in solid form. Exemplary drugs for use in the manufacture of the instant compositions include, but are not limited to, organic molecules, such as carbohydrates (including monosaccharides, oligosaccharides, and polysaccharides), steroids, nucleic acids (any form of DNA, including genes, cDNA, or RNA, or a fragment thereof), nucleotides, nucleosides, oligonucleotides (including antisense oligonucleotides), lipids, immunosuppressants, antioxidants, anesthetics, chemotherapeutic agents, steroids (including retinoids), antibiotics, antivirals, antifungals, antiproliferatives, anticoagulants, antiphotoaging agents, antimucosals, melanotropic peptides, nonsteroidal and steroidal anti-inflammatory compounds, antipsychotics, and radiation absorbers, including UV-absorbers, chemotherapeutic agents, anti-nausea medication, anti-infectives such as nitrofurazone, sodium propionate, antibiotics, including penicillin, tetracycline, oxytetracycline, chlorotetracycline, bacitracin, nystatin, streptomycin, neomycin, polymyxin, gramicidin, chloramphenicol, erythromycin, and azithromycin;

sulfonamides, including sulfacetamide, sulfamethizole, sulfamethazine, sulfadiazine, sulfamerazine, and sulfisoxazole, and anti-virals including idoxuridine; antiallergenics such as antazoline, methapyritene, chlorpheniramine, pyrilamine prophenpyridamine, hydrocortisone, cortisone, mometasone, hydrocortisone acetate, dexamethasone, dexamethasone 21-phosphate, fluocinolone, triamcinolone, medrysone, prednisolone, prednisolone 21-sodium succinate, and prednisolone acetate; desensitizing agents such as grass and ragweed pollen antigens, tree and hay fever pollen antigens, house dust mite antigens and milk antigen; vaccines such as those for smallpox, yellow fever, distemper, hog cholera, chicken pox, antivenom, scarlet fever, diphtheria toxoid, tetanus toxoid, pigeon pox, whooping cough, influenza, rabies, mumps, measles, poliomyelitic, and Newcastle disease; decongestants such as phenylephrine, naphazoline, and tetrahydrazoline; miotics and anticholinesterases such as pilocarpine, esperine salicylate, carbachol, diisopropyl fluorophosphate, phospholine iodide, and demecarium bromide; parasympatholytics such as atropine sulfate, cyclopentolate, homatropine, scopolamine, tropicamide, eucatropine, and hydroxyamphetamine; sympathomimetics such as epinephrine; sedatives and hypnotics such as pentobarbital sodium, phenobarbital, secobarbital sodium, codeine, (a-bromoisovaleryl) urea, carbromal; CNS stimulants or depressants, including opioids, such as morphine, methadone, etorphine, levorphanol, fentanyl, sufentanil, [D-Ala(2)N-MePhe(4)Gly-ol(5)]enkephalin, (DAMGO), butorphanol, buprenorphine, naloxone, naltrexone, (Cys(2), Tyr(3), Orn(5), Pen(7)-amide (CTOP), diprenorphine, β-funaltrexamine, naloxonazine, nalorphine, pentazocine, nalbuphine, naloxone benzoylhydrazone, bremazocine, ethylketocyclazocine, U50,488, U69,593, spiradoline, nor-binaltorphimine, naltrindole, [d-Pen2,D-Pen5]enkephalin (DPDPE), [D-Ala², Glu⁴]deltorphin, D-Ser-Leu-enkephalin-Thr (DSLET), Met-enkephalin, Leu-enkephalin, β-endorphin, dynorphin A, dynorphin B, α-neoendorphin, heroin, hydromorphone, oxymorphone, levallorphan, codeine, hydrocodone, oxycodone, and nalmefene; psychic energizers such as 3-(2-aminopropyl) indole acetate and 3-(2-aminobutyl) indole acetate; tranquilizers such as reserpine, chlorpromayline, and thiopropazate; hormones, including androgenic steroids such as methyl-testosterone and fluorymesterone, estrogens such as estrone, 17-β-estradiol, ethinyl estradiol, and diethyl stilbestrol, and progestational agents such as progesterone, megestrol, melengestrol, chlormadinone, ethisterone, norethynodrel, 19-norprogesterone, norethindrone, medroxyprogesterone and 17-β-hydroxy-progesterone; humoral agents such as the prostaglandins, for example PGE₁, PGE₂ and PGF₂; antipyretics such as aspirin, sodium salicylate, and salicylamide; antispasmodics such as atropine, methantheline, papaverine, and methscopolamine bromide; antimalarials such as the 4-aminoquinolines, 8-aminoquinolines, chloroquine, and pyrimethamine, antihistamines such as diphenhydramine, dimenhydrinate, tripelennamine, perphenazine, and chlorphenazine; cardioactive agents such as dibenzhydroflume thiazide, flumethiazide, chlorothiazide, and aminotrate; nutritional agents such as vitamins, including vitamin C and vitamin E, natural and synthetic bioactive peptides and proteins, including nucleoproteins, mucoproteins, lipoproteins, glycoproteins, small molecules linked to proteins, growth factors, cell adhesion factors, cytokines, and biological response modifiers; and combinations thereof.

The present invention also provides methods of making a sterilized gel composition. In various embodiments, the method comprises providing a gelling agent. In some embodiments, the gelling agent is provided in solid form. For example, the method can comprise forming a mixture of a first polysaccharide and optionally a second polysaccharide, such as a mixture of a solid hydroxyethyl cellulose and solid Kappa-carrageenan. The gelling agent can be sterilized through any sterilization methods known in the art. For example, the gelling agent can be sterilized through gamma irradiation, steam sterilization, heat sterilization, and combinations thereof. As discussed above, the gelling agent can comprise a 1:1 ratio of hydroxyethyl cellulose to Kappa-carrageenan; however, alternative ratios can be used based on the desired application of the final gel product. In addition, the gelling agent can comprise alternative components and ratios thereof. In certain embodiments, the gelling agent can comprise only one polysaccharide.

In various embodiments, the method can further comprise providing at least one propellant precursor mixed with the gelling agent, wherein the at least one propellant precursor is in solid form and substantially dry (e.g., has a moisture content of less than about 0.5%). In certain embodiments, the at least one propellant precursor is an acid component, as described in more detail above.

The method can further comprise solubilizing the solid mixture with sterile water. The ratio of water to gelling agent can be about 0.25:1 to about 1:0.25 based on volume or weight percent. In a preferred embodiment, the ratio of water to gelling agent can be about 1:1 based on volume or weight percent. In various embodiments, the solid mixture can be solubilized with a solution comprising at least one of a surfactant, an active ingredient, and a propellant precursor.

In various embodiments, upon use, an appropriate amount of propellant or propellant precursors can be added to the gelling agent solution (e.g., hydroxyethyl cellulose/Kappa-carrageenan solution) such that a sterile gas is produced which in turn causes the gel to foam. This may be accomplished, for example, by providing the composition with a first propellant precursor and then adding a second, reactive propellant precursor near or at the time of administration of the composition. In certain embodiments, the solid gelling agent or the gelling agent solution can be mixed with a first propellant precursor (e.g., an acid component) before packaging the gel composition for later use. The first propellant precursor can be in solid form, for example, when pre-mixed with the gelling agent. In such a case, a physician can, for example, mix the base component into the gel composition upon use. For example, in some embodiments where the solid composition mixture comprises at least one propellant precursor (e.g., an acid component) mixed with a gelling agent, the method can further comprise mixing an aqueous solution of a second propellant precursor (e.g., an aqueous solution of a base component) with the solid mixture.

In various embodiments of the present invention, the final gel composition can comprise (by weight of the total composition) about 20% to about 60% gelling agent; about 20% to about 60% water; about 1% to about 10% base component; and about 0.1% to about 5% acid component. In a preferred embodiment, the final gel composition can include about 40% gelling agent, about 40% water, about 5-6% base component, and about 1-2% acid component. The remaining ingredients of the gel composition can include at least one active ingredient and optionally a surfactant. After adding one or more propellant precursors (e.g., a mixture of ascorbic acid and sodium bicarbonate) to the gelling agent (e.g., a hydroxyethyl cellulose/Kappa-carrageenan solution), CO₂ gas can be produced. As a result of the in situ reaction that forms CO₂, there can be very little or no propellant precursor (e.g., acid component and/or base component) remaining in the final gel-foam product that is administered. As such, the composition may be characterized in relation to a storage form (e.g., including one or more propellant precursors) and an administration form (e.g., including a propellant and substantially no precursor propellant). The terms “substantially no precursor propellant”, “substantially no acid”, and “substantially no base” can particularly mean less than 0.5%, less than 0.1%, or less than 0.01% by weight based on the weight of the respective composition.

A sinus packing method is also provided herein wherein the method can comprise introducing into a sinus cavity a composition comprising a gelling agent that includes a first polysaccharide and optionally a second polysaccharide. In various embodiments, the second polysaccharide can be Kappa-carrageenan. It is noted that such a method can encompass any of the compositions described herein.

A kit for the preparation of a composition for use in a sinus cavity is also provided herein. The kit can comprise one or more containers containing a first polysaccharide in solid form, a second polysaccharide in solid form, wherein the second polysaccharide is Kappa-carrageenan, and one or both of an acid and a base. The kit can further include instructions for mixing the contents of the one or more containers with an aqueous medium to form a gel that includes a propellant. In various embodiments, the kit can include an acid in solid form that is separate from the gelling agent (i.e., the first polysaccharide in solid form and the second polysaccharide in solid form) before mixing. For example, the acid in solid form can be in a first syringe and the gelling agent in solid form can be in a second syringe. In some embodiments, an acid in solid form is mixed in a single container (e.g., a syringe) with the first polysaccharide in solid form and the second polysaccharide in solid form prior to forming the gel with a propellant. The instructions can include information on an amount of sterile water necessary to solubilize the acid and the first and second polysaccharides. Each of the acid, the first polysaccharide and the second polysaccharide can be solubilized separately, or together.

The kit can further comprise a base component. In certain embodiments, the base component can be in solid form. In some embodiments, the base component can be in a solution comprising sterile water and the base component. In various embodiments, the acid can be separate from the base component prior to mixing. For example, the base component in solid form can be stored in a syringe with the gelling agent (i.e., the first polysaccharide and the second polysaccharide) in solid form, but separate from the acid component. In some embodiments, the base component can be stored in its own container and can be in solid form or in a solution.

In various embodiments, the instructions can include mixing a solution comprising sterile water and a base component with the contents of the one or more containers, such that the gel is formed and the acid reacts with the base component to form the propellant in the gel. In certain embodiments, the instructions can include adding a surfactant and/or an active ingredient to a solution comprising sterile water and a base component. As described above, the kit can provide the base component. Alternatively, the base component can be separate from the kit and the kit can include instructions as to possible base components suitable for use in the invention and amounts thereof.

In some embodiments, a base component in solid form can be mixed with the acid component and the gelling agent before solubilizing the solid ingredients and the instructions can simply provide the amount of sterile water to be mixed with the gelling agent, the acid component and the base component. The instructions can further provide an amount of surfactant and/or active ingredient to be mixed with the sterile water before solubilizing the solid ingredients.

This final sterilized gel product can be used, for example, as a drug delivery/packing device for the sinus cavity. The application of this gel can be achieved by use of a syringe and possibly an apparatus intended to navigate the nasal cavity up to the sinus. In some embodiments, a volume of the gel composition in a range of about 10-15 mL can be provided, to be stored and delivered at room temperature.

### EXPERIMENTAL

A number of examples are described below to demonstrate the types of compositions that can be used t in the manner described herein. It will be understood that various details of the invention may be changed without departing from the scope of the invention. Furthermore, the foregoing description is for the purpose of illustration only, and not for the purpose of limitation, the invention being defined by the claims.

### Example 1

A gel composition capable of use as a drug delivery device in the sinus cavity is prepared. Solid components of the composition are solubilized in 9.2 ml of sterile water, and 0.8 ml of 8.4% sodium bicarbonate is added. The concentrations of the several components in the composition as fainted are as follows (based on the total weight of the composition at the time of mixing): hydroxyethyl cellulose (5% by weight); Kappa-carrageenan (1-5% by weight as desired by the formulating physician); ascorbic acid (2% by weight); sodium bicarbonate (0.67% by weight); and the balance water. Upon combination of the individual components, a sterile gas (CO₂) is formed such that the gel composition is in the form of a foam. Because the ascorbic acid and sodium bicarbonate form a reaction to create the carbon dioxide, there will be substantially no ascorbic acid or sodium bicarbonate in the final product after it is inserted in the sinus cavity.

### Example 2

Testing was carried out to identify a gelling agent with sufficient surface tension to contain air in pressurized micelles.

Hydroxyethyl cellulose, its analogue hydroxypropyl cellulose, and carboxymethyl cellulose were tested initially. Hydroxyethyl cellulose and hydroxypropyl cellulose behaved similarly in a water solution such that they each formed a gel with good thickness in water; however, both solutions had a texture that can be relatively difficult to work with. As such, an ethyl alcohol gel of hydroxyethyl cellulose or hydroxypropyl cellulose was tested. An ethyl alcohol gel of hydroxyethyl cellulose or hydroxypropyl cellulose provided satisfactory thickness requirements without the difficult texture associated with a water-based gel; however, alcohol based gels can raise concerns about patient comfort during use. Gels formed from carboxymethyl cellulose did not meet the thickness requirements. Similarly, a 1:1 blend of hydroxyethyl cellulose and carboxymethyl cellulose was tested in water, but the blend did not wet efficiently and thus did not meet thickness requirements.

Because of the failure of the initial candidate chemicals, carrageenan was tested as a gelling agent. First, an extract of Irish moss with unknown carrageenan composition was tested at three concentrations. None of them were found to gel noticeably. Upon further testing, it is discovered that an 18 percent by weight or volume mixture gelled well and produced a foam of excellent consistency, but the color was not satisfactory.

K-carrageenan was used as a gelling agent and the gel product was tested for gel concentration and consistency. Use of K-carrageenan in a range of 5%-8% (w/v) produced a gel with a workable stiffness but an unsatisfactory texture. To ameliorate this problem, a gel was made with 5% hydroxyethyl cellulose and 5% κ-carrageenan, which proved to have an improved texture and a thickness that satisfied the application requirements.

A final concentration range of 8%-20% (w/v) with a 1:1 mixture of κ-carrageenan and hydroxyethyl cellulose was established as being preferred. Concentrations of 5% hydroxyethyl cellulose/1% k-carrageenan, and 5% hydroxyethyl cellulose/3% k-carrageenan were also tested. The 5%/5% concentration was found to be preferable; however, the other mixtures exhibited qualities that could be desirable in different situations.

### Example 3

A gel composition for use as a drug delivery and packing device in the sinus cavity was produced.

A concentration range of 8%-20% (w/v) with a 1:1 mixture of κ-carrageenan and hydroxyethyl cellulose was selected as a gelling agent. CO₂ derived from the reaction of an acid with sodium bicarbonate (NaHCO₃) was chosen as a propellant. The acid used was ascorbic acid. About 150 mg-200 mg of ascorbic acid and 0.80 mL of commercially available 8.4% sodium bicarbonate, which evolves CO₂ upon mixing, was mixed with the κ-carrageenan and hydroxyethyl cellulose mixture. Approximately 8 mL of the so-generated CO₂ gas was mixed with the hydroxyethyl cellulose/κ-carrageenan mixture, which had been partially solvated in sterile water. This total mixture was mixed vigorously producing a foam of approximately 15 mL in volume. An active pharmaceutical ingredient (API) in solution can be used in place of water to impart drug delivery functionality.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed:

1. A gel composition for use in a sinus cavity comprising:
a gelling agent comprising a first polysaccharide;
a second polysaccharide, wherein the second polysaccharide is Kappa-carrageenan;
optionally a propellant in the form of a sterile gas; and
optionally at least one active ingredient.

2. The gel composition according to claim 1, wherein the first polysaccharide is selected from the group consisting of hydroxyethyl cellulose, hydroxypropyl cellulose, carboxymethyl cellulose, and combinations thereof.

3. The gel composition according to claim 1, wherein the first polysaccharide is present in an amount of at least about 10 dry weight percent.

4. The gel composition according to claim 1, wherein the Kappa-carrageenan is present in an amount of at least about 10 dry weight percent.

5. The gel composition according to claim 1, wherein the propellant is present and comprises carbon dioxide gas.

6. The gel composition according to claim 5, wherein the carbon dioxide gas is a reaction product of an acid component and a base component.

7. The gel composition according to claim 6, wherein the base component comprises sodium bicarbonate (NaHCO₃).

8. The gel composition according to claim 6, wherein the acid component comprises at least one of ascorbic acid, citric acid, and sorbic acid.

9. The gel composition according to claim 1, wherein the gel composition further comprises at least one surfactant.

10. The gel composition according to claim 9, wherein the surfactant is present in an amount of about 0.0001 to about 5.0 dry weight percent.

11. A method of providing a sterilized gel composition comprising:
providing a gelling agent mixture comprising at least a first solid polysaccharide and second solid polysaccharide, wherein the second solid polysaccharide is Kappa-carrageenan;
sterilizing the gelling agent;
solubilizing the gelling agent with sterile water to form a gelling solution; and
adding a propellant to the gelling solution to form a sterilized gel composition.

12. The method of claim 11, wherein the propellant comprises carbon dioxide gas.

13. The method of claim 12, wherein the carbon dioxide gas is a reaction product of an acid component and a base component, and wherein the base component and the acid component are initially present in the gel composition in stoichiometric amounts such that the amount of base component is sufficient to react with the desired amount of acid component.

14. The method of claim 11, wherein the step of adding a propellant to the gelling solution comprises mixing an acid precursor in powder form and a base precursor in powder form with the gelling agent mixture before solubilizing the gelling agent mixture.

15. The method of claim 11, wherein the step of adding a propellant to the gelling solution comprises mixing an acid precursor in powder form with the gelling agent mixture and mixing a base component in powder form with the sterile water to form a base precursor solution, wherein the base precursor solution is used to solubilize the gelling agent mixture.

16. A composition for use in a sinus cavity comprising:
a first polysaccharide in solid form;
a second polysaccharide in solid form, wherein the second polysaccharide is Kappa-carrageenan; and
optionally one or both of an acid in solid form and a base in solid form.

17. A kit for the preparation of a composition for use in a sinus cavity comprising:
one or more containers containing a first polysaccharide in solid form, a second polysaccharide in solid form, wherein the second polysaccharide is Kappa-carrageenan, and one or both of an acid and a base; and
instructions for mixing the contents of the one or more containers with an aqueous medium to form a gel including a propellant.

18. The kit of claim 17, comprising an acid in solid form and a base, wherein the acid is separate from the base.

19. The kit of claim 17, comprising a base, wherein the base is an aqueous solution.

20. The kit of claim 17, comprising an acid in solid form, wherein the acid is separate from the first polysaccharide in solid form and the second polysaccharide in solid form.

21. The kit of claim 17, comprising an acid in solid form, wherein the acid is mixed in a single container with the first polysaccharide in solid form and the second polysaccharide in solid form.

22. The kit of claim 17, comprising an acid in solid form, wherein the instructions include mixing the contents of the one or more containers with an aqueous solution of a base, such that the gel is formed and the acid reacts with the base to form the propellant in the gel.

23. A sinus packing method comprising introducing into a sinus cavity a composition comprising: a first polysaccharide, wherein the first polysaccharide is hydroxyethyl cellulose; and optionally a second polysaccharide, wherein if present, the second polysaccharide is Kappa-carrageenan.
